(19) United States
(12) Patent Application Publication
(10) Pub. No.: US 2017/0136079 A1
(43) Pub. Date: May 18, 2017

(54) Herbal Composition And Process For Preparing Detoxifying Herbal Extract For Snake Bite

(71) Applicants: Gencor Pacific Organics India Pvt. Ltd., Chennai (IN); University of Madras, Chennai (IN)

(72) Inventors: S. AROUMOUGAME, Muthialpet, Puducherry (IN); N. MATHIVANAN, Chennai (IN)

(21) Appl. No.: 15/322,652

(22) PCT Filed: Jul. 2, 2015
(86) PCT No.: PCT/IN2015/000273

§ 371 Date: Dec. 28, 2016

(30) Foreign Application Priority Data
Jul. 2, 2014 (IN) 3259/CHE/2014

(51) Int. Cl. A61K 36/48

(52) U.S. Cl. CPC A61K 36/48; A61K 2236/15; A61K 2236/33; A61K 2236/17

(57) ABSTRACT

This invention relates to a herbal composition and process for preparing a detoxifying herbal extract using the stem bark of the woody liana, Dalbergia horrida for treatment of snake bite, specifically Cobra (Naja naja) and Russell's viper (Daboia russelli) in mammalians. The herbal composition can also be used to treat venomous bites of krait, bugarus caeruleus, Saw scaled viper, Echis carinaturs, Monocelled cobra, Naja kaouthia and King cobra, Oohiophagus hannah. An extract (Dalbergia horrida) can be performed utilizing an ethanol solvent. Further, the extract can be performed utilizing a methanol solvent (an alcoholic solvent) separately. The ethanol extraction and methanol extraction of the herb can be performed using a common plant extraction approach (CPEA). The concentrated ethanol and methanol solvents can be pooled together to obtain a novel detoxifying herbal extract for treatment of snake bite.

### TECHNICAL FIELD

Embodiments are generally related to field of pharmaceutics, medicine and allied industries. Embodiments are also related to herbal compositions, extraction processes and techniques. Embodiments are additionally related to herbal extracts and constituents for treating snake bite. Embodiments are particularly related to a novel herbal composition and process for preparing the detoxifying herbal extract using the stem bark of the woody liana, Dalbergia horrida for treatment of snake bite in mammalians.

### BACKGROUND OF THE INVENTION

Snake envenomation is a major cause of death and disability in most of the developing countries of Asia, particularly, India. Species variation in venom components, yield, and lethality leads to quite different clinical complexities and mortality. Typically, snake venom contains of complex mixture of enzymatic and non-enzymatic toxic compounds including, such as, for example, pro-coagulants, non-enzyme proteins, peptides, carbohydrate, amines, lipids and metal ions. Additionally, enzymes such as phospholipase A₂ (PLA₂), protease, hyaluronidase, 5′nucleotidase, ATPase, alkaline phosphomonoesterase, etc. have been reported to be associated with snake venom. Such enzymes present in the snake venom are primarily responsible for causing several biological symptoms such as, but not limited to, haemorrhage, haemolysis, defibrinogenation, inflammation, edema, necrosis, proteolysis, cardiotoxicity, neurotoxicity, myotoxicity, myonecrosis, pro-coagulation, anti-coagulation and lethality, etc.

The clinical results of envenomation of the snake can vary on various factors including, size and species of the snake, the degree of toxicity of the venom, location of the bite, and amount of venom injected by the snake. It is also reported that not all snakebites shall result in envenomation. Statistics show that almost 40-45% of snakebites are of blank bites. It is however, victims from tropical geography who are still facing huge consequences due to snakebites often. In India, it is reported that approximately 2, 50,000 snakebites are annually recorded. It is also recorded that there are approximately 52 poisonous spices of snakes including Cobra (Naja naja) and Russell's viper (Daboia russelli) found in rural India.

Anti-Snake Venom (ASV) is been widely adopted for treatment of snakebites across the globe. Anti-Snake Venom is a biological product that is well known in the art for treatment of snakebites. Anti-Snake Venom typically consists of venom neutralizing antibodies derived from a host animal, normally, horse or sheep. However, Anti-Snake Venom is unable to provide required protection against local envenomation such as, venom-induced haemorrhage, necrosis, and nephrotoxicity. To add to the complexity of the treatment, use of ASV's may lead to hypersensitivity reactions in patient.

Furthermore, serum sickness is an expected side effect of anti-venom therapy which results in inflammation of certain tissues and other symptoms. Moreover, due to geographical variation in snake venom composition, anti-venom raised against the venoms of snakes from a particular geographical origin may not be able to neutralize or prevent local effects of envenomation by snakes from others geographical locations. Another serious problem faced with the anti-venom therapy is its failure to effectively neutralize the low molecular weight and less immunogenic toxic components of the venom, which causes local haemorrhage, necrosis and tissue damage in snake bite victims. Also, anti-venom immunotherapy is a costly treatment affair which can be a cumbersome choice for patients. It is therefore, an effective treatment for snakebite is highly inevitable for treatment of snakebites.

Herbal preparation based anti-snake venoms are alternatively adapted for treatment of snakebites. Prior art methods and processes for preparing herbal preparation are also known in the art. Chinese application no. CN 93101304 describes a snake bite medicine and its preparation method which uses herb of common St. Paulswort. Another prior art application, Chinese patent application no. CN 101797313 A, discloses a Chinese herbal medicine for treatment of venomous snakebite using components of Artemisia annua. U.S. Pat. No. 4,568,546 A describes a composition effective against the symptoms of venom, such as convulsions and throat blockage, suffered by a victim of a venomous creature, which composition comprises an ethyl alcohol infusion of previously dried herbs and iron filings.

Most of the herbal compositions in the prior arts use parts of arrow head, sweet worm wood, sagittaria sagittifolia L and Philippine violet herb for preparing the anti-venom composition for treating symptoms of venom. Other prior art herbal compositions and methods for preparation that are known in the art are however complex, time consuming and ineffective for treatment of snakebites of deadly Indian snakes including Cobra (Naja naja) and Russell's viper (Daboia russelli).

Based on the foregoing, it is believed that a need exists for an improved herbal composition for snake bite which is simple, easy to use and effective. A need also exists for an improved process for preparing a novel detoxifying herbal extract using the stem bark of the woody liana, Dalbergia horrida for treatment of snake bite in mammalians, as described in greater detail herein.

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for an improved herbal composition for treatment of snakebite in mammalian.

It is another aspect of the disclosed embodiments to provide for an improved process for preparing detoxifying herbal extract for snakebite.

It is further aspect of the disclosed embodiments to provide for an improved process for preparing a novel detoxifying herbal extract using the stem bark of the woody liana, Dalbergia horrida for treatment of snake bite specifically Cobra (Naja naja) and Russell's viper (Daboia russelli).

The aforementioned aspects and other objectives and advantages can now be achieved as described herein. A herbal composition and process for preparing a detoxifying herbal extract using the stein bark of the woody liana, Dalbergia horrida for treatment of snake bite specifically Cobra (Naja naja) and Russell's viper (Daboia russelli), is disclosed herein. The herbal composition can also be used to treat venomous bites of krait, bugarus caeruleus, Saw scaled viper, Echis carinatus, Monocelled cobra, Naja kaouthia and King cobra, Oohiophagus hannah. An extract (Dalbergia horrida) can be performed utilizing an ethanol solvent. Further, the extract can be performed utilizing a methanol solvent (an alcoholic solvent) separately. The ethanol extraction and methanol extraction of the herb can be performed using a common plant extraction approach (CPEA). The concentrated ethanol and methanol solvents extract can be pooled together in order to obtain a novel detoxifying herbal extract for treatment of snakebite.

The Common Plant Extraction Approach (CPEA) described herein can be a principle extraction technique that is well known in the art for extracting the crude extract of a plant/herb. Note that the extraction technique disclosed herein should not be considered in any limited sense. A person skilled in the art can adapt any other extraction processes to achieve the required output without limiting the scope of the invention. The stem barks of D. horrida can be sliced into pieces (Approximately 1 cm size) and shade dried at room temperature. Further, a mechanized pulverizing machine can be employed to grind the dried plant slices into coarse powder in order to thereby extract the compounds using the ethanol and methanol solvents separately.

The extract can be applied topically as well as consumed orally as soon as victimized by the snake bite. If victim is moved to a safer place the extract can be used in injectable form. Also, it is recommended that the people who are moving towards snake prone areas like forest, agricultural fields etc can consume sufficient quantities of the extract of Dalbergia horrida as preventive measure to a shorter duration. The possible mechanism of neutralization of the snake venom of the species, cobra as well as Russell's viper by the presence of secondary metabolites more specifically phenolic as well as flavanodal compounds in the extract is expected to denature the antigen, the venom protein and thereby the venom protein is deactivated leading to disabled to bind to the receptors.

The solvent extraction process described herein can be performed at 1:10(W/V) ratio using a soxhlet apparatus in order to thereby separate the reddish brown colored solvent extracts using a rotoevaporator. The crude extracts obtained at both the ethanol solvent extraction process and the methanol solvent extraction process are further pooled together or used separately in order to form the herbal extract for detoxifying the snake venoms. The solvents utilized for extracting compounds as a whole herein should not be construed in any limited sense. Those skilled in the art can understand that similar kind of solvents such as hexane, ethyle acetate, acetone, chloroform, dichloromethane, etc. can also tried for preparing herbal extraction and developing the detoxifying snake venoms formulation.

The herbal extract can be dissolved in the sterile distilled water or 5% DMSO (at 200 mg-400 mg/2 ml) and the aqueous suspension is subjected to centrifuge at 2000 rpm in order to form a clear solution without any suspended particulate matter. The proposed process of preparing detoxifying herbal extract for snakebite can be an effective low cost alternative for treating snakebite of various poisonous snakes including Cobra (Naja naja) and Russell's viper (Daboia russelli) in mammalians.

### BRIEF DESCRIPTION OF THE DRAWINGS

The accompanying FIGURES, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a high level flow chart of operation illustrating operational steps of a process for preparing detoxifying herbal extract using the stem bark of the woody liana, Dalbergia horrida for treatment of snake bite, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

The embodiments now will be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

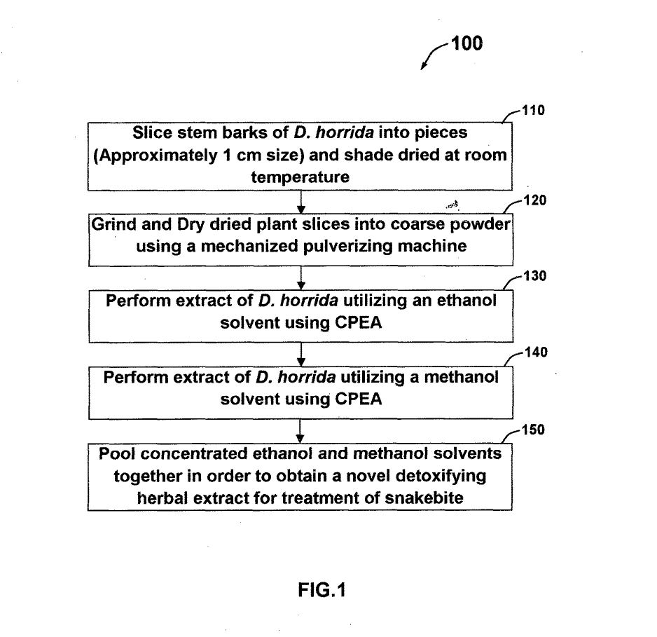

FIG. 1 illustrates a high level flow chart of operation illustrating operational steps of a process 100 for preparing detoxifying herbal extract using the stem bark of the woody liana, Dalbergia horrida for treatment of snake bite specifically Cobra (Naja naja) and Russell's viper (Daboia russelli), in accordance with the disclosed embodiments. The herbal composition can also be used to treat venomous bites of krait, bugarus caeruleus, Saw scaled viper, Echis carinatus, Monocelled cobra, Naja kaouthia and King cobra, Oohiophagus hannah. Snake bite is a common and devastating environmental and occupational disease, especially in rural areas of tropical developing countries. Snake venoms are rich in protein and peptide toxins that have specificity for a wide range of tissue receptors.

Although the full burden of human suffering attributable to snakebite remains obscure, hundreds of thousands of people are known to be envenomed and tens of thousands are killed or maimed by snakes every year. It is also statistically recorded that the snake bite is a serious medical problem in rural India and WHO has brought out snakebite as a neglected tropical disease as it accounts for the death of 50,000 people per annum in India. Considering the need for an improved treatment for snakebite, the process proposed herein can be an effective solution for treatment of snakebites.

The stem barks of D. horrida can be sliced into pieces (Approximately 1 cm size) and shade dried at room temperature, as illustrated at block 110. Further, a mechanized pulverizing machine can be employed to grind the dried plant slices into coarse powder, as disclosed at block 120. The extract (Dalbergia horrida) can be performed utilizing an ethanol solvent, as depicted at block 130. Further, the extract can be performed utilizing a methanol solvent (an alcoholic solvent) separately, as illustrated at block 140. The ethanol extraction and methanol extraction of the herb can be performed using a common plant extraction approach (CPEA). The Common Plant Extraction Approach (CPEA) described herein can be a principle extraction technique that is well known in the art for extracting the crude extract of a plant/herb. Note that the extraction technique disclosed herein should not be considered in any limited sense. A person skilled in the art can adapt any other extraction processes to achieve the required output without limiting the scope of the invention.

The concentrated ethanol and methanol solvents can be pooled together dried in order to obtain a novel detoxifying herbal extract for treatment of snakebite, as illustrated at block 150. The solvent extraction process described herein can be performed at 1:10(W/V) ratio using a soxhlet apparatus in order to thereby separate the reddish brown colored solvent extracts using a rotoevaporator. The crude extracts obtained at both the ethanol solvent extraction process and the methanol solvent extraction process are further pooled together or used separately in order to form the herbal extract for detoxifying the snake venoms.

The solvents utilized for extracting compounds as a whole herein should not be construed in any limited sense. Those skilled in the art can understand that similar kind of solvents such as hexane, ethyle acetate, acetone, chloroform, dichloromethane, etc. can also tried for preparing herbal extraction and developing the detoxifying snake venoms formulation. The herbal extract can be dissolved in the sterile distilled water or 5% DMSO (at 200 mg-400 mg/2 ml) and the aqueous suspension is subjected to centrifuge at 2000 rpm in order to form a clear solution without any suspended particulate matter.

The extract of D. horrida has showed inhibition potency of 96.87% in ELISA test against the venom of D. russelli. The extract could neutralize the venom of D. russelli completely (100%) at a dose of 48 μg in the NDPAGE study. Interestingly, the complete neutralization of 16 μg of the D. russelli venom was achieved with lesser concentration (48 μg) of the D. horrida extract. The extract was also tested against venom of N. naja. The result clearly substantiated that the percentage inhibition against the N. naja venom was 81.23, whereas the inhibition against the venom of D. russelli was 96.78. The potency of the D. horrida extract demonstrated appropriately in neutralizing the D. russelli venom at a lower concentration in the NDPAGE. The proposed process 100 of preparing detoxifying herbal extract for snakebite can be an effective low cost solution for treating snakebite of various poisonous snakes including Cobra (Naja naja) and Russell's viper (Daboia russelli).

It will be appreciated that variations of the above-disclosed and other features and functions, or alternatives thereof, may be desirably combined into many other different processes or applications. Also that various presently unforeseen or unanticipated alternatives, modifications, variations or improvements therein may be subsequently made by those skilled in the art which are also intended to be encompassed by the following claims.

1. A herbal composition for preparing a detoxifying herbal extract, said composition comprising:
stem bark of woody liana, Dalbergia horrida for treatment of snake bite specifically Cobra (Naja naja) and Russell's viper (Daboia russelli) wherein the herbal composition can also be used to treat venomous bites of krait, bugarus caeruleus, Saw scaled viper, Echis carinatur, Monocelled cobra, Naja kaouthia and King cobra, Oohiophagus hannah.

2. The herbal composition of claim 1 wherein the herbal extract (Dalbergia horrida) includes an ethanol solvent.

3. The herbal composition of claim 1 wherein the herbal extract (Dalbergia horrida) includes a methanol solvent (an alcoholic solvent) separately.

4. The herbal composition of claim 1 wherein concentrated ethanol and methanol solvents extract can be pooled together to obtain the detoxifying herbal extract for treatment of snakebite.

5. A process for preparing a detoxifying herbal extract using stem barks of woody liana, Dalbergia horrida, said process comprising:
slicing the stem barks of Dalbergia. horrida into pieces approximately 1 cm size and shade drying at room temperature; and
grounding the stem barks into coarse powder to extract compounds using ethanol and methanol solvents separately.

6. The process of claim 5 wherein the solvent extraction process can be performed at 1:10(W/V) ratio using a soxhlet apparatus to separate reddish brown colored solvent extracts using a roto-evaporator wherein crude extracts obtained at both an ethanol solvent extraction process and a methanol solvent extraction process are further combined or used separately to form the herbal extract for detoxifying the snake venoms.

7. The process of claim 5 wherein the herbal extract can be dissolved in sterile distilled water of 5% dimethyl sulfoxide (DNSO) at 200 mg-400 mg/2 ml and the aqueous suspension is subjected to centrifuge at 2000 rpm to form a clear solution without any suspended particulate matter.

8. (canceled)
